(19) United States
(12) Patent Application Publication
(10) Pub. No.: US 2025/0137195 A1
(43) Pub. Date: May 1, 2025

(54) METHOD FOR PRODUCING LEATHER USING CELL CULTURE ON MACROPOROUS POLYMERIC SCAFFOLD

(71) Applicant: FAIRCRAFT, Evry-Courcouronnes (FR)

(72) Inventors: Cesar VALENCIA GALLARDO, Saint Mande (FR); Elias AL TAWIL, Evry-Courcouronnes (FR); Sergiy KYRYACHENKO, Sartrouville (FR); Haïkel BALTI, Paris (FR)

(21) Appl. No.: 18/834,687

(22) PCT Filed: Feb. 3, 2023
(86) PCT No.: PCT/EP2023/052688

§ 371 Date: Jul. 31, 2024

(30) Foreign Application Priority Data
Feb. 4, 2022 (EP) 22305126.9

(51) Int. Cl. D06N 3/00; C12N 5/077; D06N 3/12

(52) U.S. Cl. CPC D06N 3/0043; C12N 5/0656; D06N 3/0002; D06N 3/123; D06N 3/128; C12N 2513/00; C12N 2533/30

(57) ABSTRACT

The present disclosure relates to a method for producing leather comprising the step of culturing cells on macroporous polymeric scaffold. The present disclosure also relates to leather obtainable by this method.

### TECHNICAL FIELD

The present disclosure relates to a method for producing leather comprising the step of culturing cells on macroporous polymeric scaffold. The present disclosure also relates to leather obtainable by this method.

### BACKGROUND OF THE INVENTION

Leather was ranked the first most resource intensive material used in the fashion industry, far ahead of cotton or polyester. While animal leather is appreciated for its durability and aesthetics, it carries multiple environmental challenges: intensive natural resources use, animal welfare and working conditions concerns. Moreover, for many applications, leather needs to be exempt from defaults such as scratches or insect bites. As traditional leather production relies largely on cattle farming, up to 80% of hides are discarded due unmet quality standards.

As brands are under tremendous pressure to become more ethical and sustainable, a sustainable alternative to animal leather based on cell biology is needed.

Although synthetic leather was developed to address some of these concerns, it lacks the quality of natural leather. Previous attempts to make engineered leathers have been described. For example, EP1589098 describes a method of growing fibroblasts seeded onto three-dimensional bioactive scaffolds. The scaffolds may be made from collagen waste material from a tanning process (“split”), microparticles of pure collagen, particles of collagen waste material, or synthetic scaffolds (e.g., made of polymers such as HYAFF). WO2017/184967 describes the culture of stem cells or keratinocytes on collagen or PET membranes with micropores of 4 μm. However, these culture conditions are not optimal for cell growth and collagen production.

The tanning procedure breaks down most of the cellular, molecular and extracellular matrix components. The only components that resist storage and tanning are the collagen fibers and elastin fibers that are found in the leather in its final state and are secreted by fibroblasts (Sharphouse, J. H. Leather Technician's Handbook. Leather Producer's Association. p. 104. ISBN 0-9502285-1-6). These collagen and elastin fibers are responsible for the mechanical properties of leather. For this reason, inducing the secretion of Extracellular Matrix (collagen, elastin, among others) by the network of fibroblasts proliferating into scaffolds is a key step in the development of leather.

Thus, there is still a need to develop cell culture methods that promote cell proliferation as well as the production of collagen essential for high-quality leather production.

### SUMMARY OF THE INVENTION

The inventors develop a new method for producing high-quality leather by culturing fibroblasts in vitro on a macroporous polymeric scaffold that allows efficient high cell attachment, cell culture and proliferation for long periods of time with minimal stress and damage for cells and results in a larger production of collagen by the fibroblasts.

The present invention relates to a method for producing leather comprising the steps of: a) culturing fibroblasts in vitro on a macroporous polymeric scaffold wherein said scaffold comprises macropores of diameter comprised between 60 and 500 μm, in particular between 80 and 280 μm, preferably between 100 and 280 μm, more preferably between 120 and 250 μm to obtain a tissue, b) tanning said tissue obtained in step a) thereby forming said leather.

The present invention also relates to a tissue obtainable by the culture step a) of the method of the present invention and a tanned leather obtainable by the method of the present invention.

### LEGEND FIGURES

FIG. 1: Scheme of porous scaffolds of materials based on PDMS or Poly (α-hydroxy acids) for fibroblastic cell cultivation and biofabrication of leather a) PDMS or polyester symmetric scaffold, b) PDMS or polyester asymmetric scaffold.

FIG. 2: Scheme of the symmetric PDMS or asymmetric polyester scaffolds production method by SCPL process.

FIG. 3: Surface image of porous scaffolds fabricated by SCPL process using volumetric mixtures of either PDMS (a,c,d) or polyester (b) acquired by scanning electron microscopy (SEM). a) Symmetric PDMS scaffold produced using calibrated inorganic macro porogens (150-250 μm), scale bar: 300 microns; b) Asymmetric polyester scaffold produced using calibrated organic macro porogens (mean particle size of 150 μm), scale bar: 400 microns; c) and d) PDMS scaffolds produced using calibrated organic macro porogens (mean particle size of 150 μm), scale bars: c) 400 microns and d) 100 microns.

FIG. 4: Functionalization of polymers with plasma treatments.

FIG. 5: Functionalization of plasma-activated PDMS with collagen Col (a), poly-lysine NH2 (b) or glucides or glucide-derived molecule Glu (c).

FIG. 6: Functionalization of polyester-based scaffolds: aminolysis and glucide/glucide-derived molecule immobilization.

FIG. 7: PDMS scaffold functionalized with collagen I, seeded with fibroblasts (fluorescence microscopy). Cell nuclei were stained with DAPI. Scale bar: 300 μm.

FIG. 8: 3D reconstruction (0-250 μm) of a PDMS scaffold functionalized with collagen I inoculated with fibroblasts by confocal microscopy (LEICA TCS SP8, 20X). Total cell nuclei were stained in grey, dead cells were stained in green.

FIG. 9: PDMS (a,b) and polyester (c) scaffolds were functionalized with either: a) Poly-lysine, or b,c) Poly-lysine and/or glucide/glucide-derived molecules, and seeded with fibroblasts (fluorescence microscopy). Cell nuclei stained with DAPI, Scale bars: 500 μm (a,b) and 250 μm (c).

FIG. 10: Tissue production by fibroblasts seeded into a macroporous polymeric scaffold (fluorescence microscopy). Collagen secretion from cells was observed by fluorescence microscopy using an anti-collagen I antibody (Sigma, ref. C2456). Cell nuclei were stained with DAPI (blue) and collagen I in green. Scale bar: 500 μm.

FIG. 11: Tanning process of tissue samples to obtain a tanned leather a) Tanning process carried on labware scale, b) drying of the tanned sample, and c) final result of obtained tanned leather.

FIG. 12: Surface image of tanned leather (a) and German tanned leather made by scanning electron microscopy (SEM). The thickness of the extracellular matrix cables was measured by the provided scale (microns) using ImageJ. Scale bars: 10 microns.

### DETAILED DESCRIPTION

The inventors have developed a method for producing high-quality leather using an in vitro culture step of fibroblasts on a macroporous scaffold to improve cell growth and collagen secretion which provides the stiffness, mechanical strength and resistance to abrasion found in leather.

The present disclosure relates to a method for producing leather comprising the steps of:
a) culturing fibroblasts in vitro on a macroporous polymeric scaffold wherein said scaffold comprises macropores of diameter comprised between 60 and 500 μm, preferably between 80 and 280 μm, more preferably between 100 and 280 μm, again more preferably between 120 and 250 μm to obtain a tissue,
b) tanning said tissue thereby forming said leather.

According to the method of the present disclosure, the scaffold comprises macropores of diameter comprised between 60 and 500 μm, preferably between 80 and 280 μm, more preferably between 100 and 280 μm, again more preferably between 120 and 250 μm in which the cells can penetrate, thus conferring a biological environment favoring the proliferation and the function of the fibroblasts.

According to the present disclosure, the term “scaffold” refers to a tridimensional support which provides physical and structural support for cells and allowing tissue formation.

For the purposes of the present disclosure, by the term “pore diameter” is intended the diameter average mean of the pore as measured with Scanning Electron Microscopy (SEM) by averaging the diameter of 5 to 30 pores measured using the ImageJ software (Rasband, W. S., ImageJ, U. S. National Institutes of Health, Bethesda, Maryland, USA, https://imagej.nih.gov/ij/, 1997-2018).

To maintain the mechanical structure of the scaffold while providing a suitable biological environment for cell proliferation, the macroporous scaffold preferably has a degree of porosity from 65% to 98%, in particular from 70 to 98%, 75 to 98%, more preferably 80% to 95%.

For the purposes of the present invention, by the term “degree of porosity” or “void fraction” is intended to mean the percentage of the volume of the pores relative to the volume of the material, as measured according to a gravimetric method (Guarino V, et al. 2008 Sep;29(27):3662-70). The volume of the sample was calculated from the measurements of the sample dimensions. Measurement of the mass of the sample allowed to deduce the sample density. For each type of sample, the density (ρSc) was the average value obtained from 4 samples. Porosity of the scaffold was then calculated using the formulae:

$$Porosity = ( 1 - ( ρ Sc / ρ Ppoly ) ) : 100$$

where ρPpoly is the density of the polymer according to the manufacturer's database.

Macropore formation in the scaffold can be accomplished using several techniques well-known in the art, such as solvent casting and particulate leaching (SCPL) or phase inversion method.

The scaffold according to the present disclosure can be engineered to present a thickness that is adapted for the leather synthesis. In particular, the scaffold can have a thickness from 0.5 to 2.5 mm.

The scaffold according to the present disclosure can be engineered to present a thickness that is adapted for the creation of different biomaterials. In particular, the scaffold can have a thickness from 0.25 to 4.5 mm, particularly from 0.5 to 2 mm.

According to one particular embodiment, the polymer support does not have a fibrous structure.

In a particular embodiment, the polymers that can be used in the scaffold may be organosilicon based polymer such as polydimethylsiloxane (PDMS), Poly (α-hydroxy acids) or mixture thereof.

In a particular embodiment, the polymers that can be used in the scaffold may be polymeric organosilicon compounds such as PDMS which presents biocompatibility and long-term stability in contact with biological material. Moreover, the high solubility of oxygen in the PDMS matrix allows the oxygen to be supplied to the cell and to prevent hypoxia (Merkel, T.C., et al. 2000, J. Polym. Sci. B Polym. Phys., 38: 415-434). Moreover, polymer scaffold with high oxygen permeability promotes gas exchange, nutrients and the removal of waste associated with cell metabolism and are particularly suitable for improving fibroblastic cell proliferation and function in vitro. In a particular embodiment, said PDMS is SYLGARD™ 184 PDMS (Dow).

In a particular embodiment, when said scaffold is composed of organosilicon based polymer such as PDMS, macropore formation can be accomplished by solvent casting and particulate leaching (SCPL). For example, the SCPL method can be performed by mixing the polymer with a catalyst. Particulates, herein named porogen with specific dimensions are then added to the polymer/catalyser solution, mixed and casted onto a support to allow reticulation. When the polymer reticulates, it creates a structure of composite material consisting of the porogen together with the polymer. The composite material is then placed in a bath which dissolves the porogen, leaving a porous scaffold. The porogen solvent that is used is selected for its characteristic of dissolving the porogen and for its characteristic of not being a solvent for the polymer.

Examples of suitable porogen include inorganic salt crystals such as sodium chloride crystals, potassium chloride, sodium sulfate and amorphous materials such as poly(ethylene glycol) (PEG), polyvinylpyrrolidone, crystals of sucrose, gelatin spheres, paraffin spheres.

Examples of porogen solvents include chloroform, tetrahydrofuran, dimethylsulfoxide, methanol and water.

The solvent for the polymer may in particular be N,N-dimethylformamide, N-methylpyrrolidone, dimethylacetamide, dimethyl sulfoxide, chloroform and mixtures thereof.

The catalysts may be any polymeric organosilicon catalysts known in the art, in particular PDMS catalysts such as platinum or palladium catalysts.

The concentration of porogen in the solution of polymer may in particular be from 50% to 90%, in particular from 75% to 85% by weight relative to the weight of the polymer.

Said scaffold obtained by SCPL method is a symmetric scaffold with interconnected macropores of diameter comprised preferably between 100 to 280 μm distributed throughout the scaffold.

By symmetric scaffold, it is intended a scaffold wherein macropores of diameter comprised between 60 and 500 μm, preferably between 80 and 280 μm, more preferably between 100 and 280 μm, again more preferably between 120 and 250 μm are homogeneously distributed throughout the thickness of the scaffold, and in particular wherein said scaffold has two macroporous faces, in particular wherein said faces both comprise macropores of diameter comprised between 60 and 500 μm, preferably between 80 and 280 μm, more preferably between 100 and 280 μm, again more preferably between 120 and 250 μm.

In another particular embodiment, the polymers that can be used in the scaffold may be polyester derived poly (α-hydroxy acids).

Non-limiting examples of polyester derived poly (α-hydroxy acids), in particular biosourced and biodegradable polyesters that can be used to form the scaffold are homopolymers and copolymers of hydroxy acids, such as polylactic acid (PLA), polyglycolic acid (PGA), lactic and glycolic acid copolymers (PLGA), poly(3-hydroxybutyrate), poly(4-hydroxybutyrate), poly(3-hydroxyvalerate), poly(5-hydroxyvalerate), poly(3-hydroxypropionate), poly(3-hydroxyhexanoate), poly(3-hydroxyoctanoate), poly(3-hydroxyoctodecanoate); polycaprolactone; homopolymers and copolymers of poly(butylene succinate) and of poly(butylene adipate); and polyhydroxyalkanoates (PHA) and their derivatives polyhydroxyesters of 3-, 4-, 5-, and 6 hydroxyalkanoic acid and mixtures thereof.

Polyesters have interesting properties from a mechanical point of view, they are compatible with cell culture. They are biosourced since they are produced from agricultural waste and are fully biodegradable. However, polyesters are hydrophobic and impermeable to oxygen and do not allow the diffusion of molecules after reticulation.

Thus, to promote gas exchange, said oxygen impermeable polymer scaffold can further comprise nanopores and micropores which have a diameter of less than 20 μm, in particular from 0.1 μm to 10 μm, more particularly from 2 to 8 μm.

Said scaffold composed of oxygen impermeable polymer such as polyester polymer and comprising micropores can be an asymmetric scaffold or symmetric scaffold.

The polyester polymer symmetric scaffold according to the present disclosure can be accomplished by any methods known by one skilled in the art. As non-limiting example by the polymer symmetric scaffold can be obtained by a combination of one phase inversion process (e.g. NIPS) which allows the formation of micropores promoting gas and nutrients exchange, and a solvent casting and particulate leaching (SCPL) process to form macropores allowing cell growing and tissue formation.

Phase inversion are method well-known in the art and included for examples Non-solvent Induced Phase Separation Process (NIPS), Thermally Induced Separation Process (TIPS), Vapor Induced Phase Separation process (VIPS), and Polymerization Induced Phase Separation (PIPS).

In particular, the scaffold can be produced by the steps of: preparing a solution of said polymer comprising said polymer and at least one solvent for the polymer as described above; adding a solid porogen to the ready solution of the polymer under homogeneous stirring; pouring said solution containing the polymer and the porogen onto the solid support; and introducing said solution of the polymer into non-solvent solution (water), and finally immersing the porogen/polymer composite in a porogenic solvent to dissolve porogen.

Examples of suitable porogen include inorganic salt crystals such as sodium chloride crystals or potassium chloride and amorphous materials such as poly(ethylene glycol) (PEG), polyvinylpyrrolidone, crystals of sucrose, gelatin spheres and paraffin spheres.

The concentration of porogen in the solution of polymer may in particular be from 50% to 98%, in particular from 80% to 95% by weight relative to the weight of the polymer.

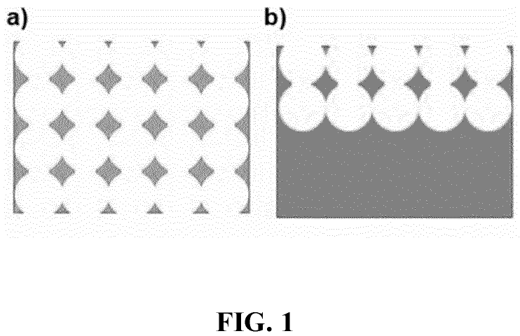

The formation of micropores of different sizes is observed (NIPS) when phase inversion happens at the polymer interface with the non-solvent solution, and the formation of macropores is observed following the SCPL process. The polyester scaffold obtained by the combination of NIPS process and SCPL process is a symmetric scaffold which has a network of interconnected macropores of diameter comprised between 60 and 500 μm, preferably between 80 and 280 μm, more preferably between 100 and 280 μm, again more preferably between 120 and 250 μm and micropores which have a diameter of less than 20 μm, in particular from 0.1 μm to 15 μm, more particularly from 2 to 8 μm distributed throughout the thickness of the scaffold as represented in FIG. 1a. In particular, said polyester symmetric scaffold presents two macroporous faces, in particular wherein said two faces both comprise macropores of diameter between 60 and 500 μm, preferably between 80 and 280 μm, more preferably between 100 and 280 μm, again more preferably between 120 and 250 μm.

According to the methods described above, the solid substrate on which said solution of the polymer is deposited can in particular be made of glass, metal or plastic resistant to solvents, such as polytetrafluoroethylene (Teflon®), nylon 6,6 or poly(ethylene terephthalate).

In another particular embodiment, said polyester scaffold can be obtained by the combination of SCPL and phase inversion method selected from the group consisting of: TIPS, VIPS and PIPS.

TIPS is a process wherein a polymer solution is formed at high temperature with a high-boiling point solvent and cooled to induce phase separation and polymer solidification. The microporous scaffolds are obtained after the extraction of the diluent.

In the VIPS process, a cast film consisting of polymer and solvent is exposed to a vapor atmosphere of nonsolvent molecules, typically water. The precipitation of the polymer occurs due to the penetration of the vapor into the film, which eventually forms a symmetric porous scaffold without a dense skin layer. The thermodynamic properties of the casting solution in the NIPS and VIPS methods are almost similar, suggesting that the VIPS method should produce scaffolds with morphologies like those produced using the NIPS method.

PIPS process is a phase separation which occurs in a multicomponent mixture induced by the polymerization of one or more components. The increase in molecular weight of the reactive component renders one or more components to be mutually immiscible in one another, resulting in spontaneous phase segregation. The morphology of the final phase separated structures are generally random owing to the stochastic nature of the onset and process of phase separation.

In another particular embodiment, the polyester polymer scaffold is an asymmetric scaffold and can be accomplished by any methods known by one skilled in the art. Said asymmetric scaffolds are obtained by the solvent casting and particulate leaching (SCPL) process to form macropores allowing cell growing and tissue formation.

In particular, the scaffold can be produced by the steps of: preparing a solution of said polymer comprising said polymer and at least one solvent for the polymer as described above; adding a solid porogen to the ready solution of the polymer under homogeneous stirring; and letting the solvent evaporate slowly to reticulate the polymer, and finally immersing the porogen/polymer composite in a porogenic solvent to dissolve porogen.

An asymmetric macroporous scaffold is formed when solvent evaporation happens and when the porogen is dissolved into the porogenic solvent. The rapid evaporation of the solvent in the upper face gives a dense nanoporous face and the slow evaporation of the solvent in the depth of the polymeric solution gives micropores. The porogen leaching gives the macropores.

Examples of suitable porogen include inorganic salt crystals such as sodium chloride crystals or potassium chloride and amorphous materials such as poly(ethylene glycol) (PEG), polyvinylpyrrolidone, crystals of sucroses, gelatin spheres, paraffin spheres.

The concentration of porogen in the solution of polymer may in particular be from 500% to 900%, in particular from 700% to 900% by weight relative to the weight of the polymer.

The polyester asymmetric scaffold obtained by the SCPL process is an asymmetric scaffold which has a network of interconnected macropores of diameter comprised between 60 and 500 μm, preferably between 80 and 280 μm, more preferably between 100 and 280 μm, again more preferably between 120 and 250 μm and micropores which have a diameter of less than 10 μm, in particular from 0.1 μm to 5 μm, more particularly from 1 to 5 μm distributed throughout the thickness of the scaffold as represented in FIG. 1b. In particular, said polyester asymmetric scaffold presents a macroporous face and a nanoporous face, more particularly which presents a first face comprising macropores of diameter comprised between 60 and 500 μm, preferably between 80 and 280 μm, more preferably between 100 and 280 μm, again more preferably between 120 and 250 μm and a second opposite face comprising only nanopores of diameter below 10 nm, preferably below 5 nm.

In order to functionalize the surface of the scaffolds designed as described above and increase the cytocompatibility of the scaffold, a bioactive molecule can be grafted at the surface of said scaffold. Said bioactive molecule can be chosen from polysaccharides such as cellulose, pectin, pullulan, keratan, hyaluronan, chondroitin sulfates, chitosan and heparin; proteins such as fibrinogen and collagen, in particular soluble collagen; peptides such as those known for their cell adhesion capacity, for instance those containing the arginine-glycine-aspartic acid (RGD) or arginine-glycine-aspartic acid-serine (RGDS) sequence; and mixtures thereof. The bioactive molecules can be grafted by any methods well-known in the art.

According to one particular embodiment, the bioactive molecules are collagen. Indeed, collagen allows cell adhesion as collagen specifically interacts with integrin receptors expressed at the surface of skin-derived cells. In order to graft collagen at the surface of the scaffold, in a particular embodiment, said scaffold can be treated with plasma generated in air or oxygen, to add electronegative silanol groups. Collagen is then grafted at the surface of the scaffold by immersing said scaffold in a solution comprising collagen, followed by rinsing, preferably with phosphate buffered-saline solution (PBS).

According to one preferred embodiment, the bioactive molecules can be glucides and/or glucide derived molecules such as glycosaminoglycans (e.g., heparin sulfate, heparin, chondroitin sulfate, dermatan sulfate, keratan sulfate, hyaluronic acid). By virtue of its unique biophysicochemical properties (viscoelasticity, high water retention capacity, capacity to interact specifically or nonspecifically with various proteins), glycosaminoglycans play a fundamental role in extracellular matrix organization and homeostasis.

In order to graft glucides and/or glucide-derived molecules (e.g. glycosaminoglycan) at the surface of said scaffold, the scaffold can be firstly pretreated to bring positive charges, preferably positive amine group to the surface of said scaffold before being functionalized with negatively charged molecules. Said positive amine group may be added by treating said scaffold with plasma generated in oxygen or air and immersing said scaffold in polylysine solution, by treating said scaffold with plasma generated in azote or by aminolysis reaction. Once the surface of the scaffold has been treated to be positively charged, said scaffold can be functionalized with glucides and/or glucide-derived molecules by immersing the scaffold in a solution comprising the said glucides and/or glucide-derived molecules, followed by rinsing, for example with ultrapure water.

According to a preferred embodiment, said positive amine group is added by aminolysis reaction of the polymer using a solution of at least one aliphatic α,ω-diamine. The aminolysis reaction can in particular be carried out by immersing the polymer scaffold in a solution comprising aliphatic α,ω-diamine, preferably by immersing said scaffold with 1,6-hexanediamine solution in propanol or water and mixtures thereof, followed by rinsing for example with ultrapure water.

According to the method of the present disclosure, fibroblasts are seeded onto the polymer scaffold as described above and cultured under suitable growth conditions, in particular in a medium which allows the proliferation and the induction of the secretion of extracellular matrix such as different types of collagen and elastin fibers.

Fibroblasts according to the present disclosure is a cell that synthesizes extracellular matrix such as collagen, elastin, glycoproteins and non-proteoglycan polysaccharides among other extracellular matrix components. Fibroblasts can be mammalian or non-mammalian fibroblasts. The cells can be a single cell type or a combination of cell types. As the only components that resist to the storage and tanning processes are the collagen and elastin fibers that are secreted by fibroblasts, in a preferred embodiment, fibroblasts are the only cells cultured on the scaffold according to the method of the present disclosure.

Numerous culture mediums are available commercially and are well-known to the person skilled in the art. This medium may be a minimum medium particularly comprising mineral salts, amino acids, vitamins and a carbon source essential to cells and a buffer system for regulating pH. The basal medium able to be used in the method according to the invention includes, for example, but are not limited to, MEM medium, DMEM/F12 medium, DMEM medium RPMI medium, Ham's F12 medium, IMDM medium and KnockOut™ DMEM medium (Life Technologies). Depending on the medium used, it may be necessary or desirable to add glutamine, ascorbic acid, growth factor, one or more antibiotics such as streptomycin, penicillin and/or anti-mycotic.

In another aspect, the present disclosure also relates to a tissue (also named in-vitro skin) obtainable by the cell culture step a) as described above, preferably the tissue comprises fibroblastic cells cultured on a macroporous polymeric scaffold as described above comprising macropores of diameter comprised between 60 and 500 μm, preferably 80 to 280 μm, more preferably between 100 and 280 μm, more preferably between 120 and 250 μm.

In a particular embodiment, said scaffold comprises at least one polymer such as polymeric organosilicon compound such as polydimethylsiloxane (PDMS) or biodegradable polyester.

In a preferred embodiment, said tissue comprises a scaffold with macropores interconnected with micropores, preferably said micropores having a diameter of less than 20 μm, preferably less than 10 μm.

In a particular embodiment the tissue comprises an asymmetric scaffold comprising macropores of diameter comprised between 80 and 280 μm, more preferably between 100 and 280 μm, again more preferably between 120 and 250 μm and micropores which have a diameter of less than 10 μm, in particular from 0.1 μm to 5 μm, more particularly from 1 to 5 μm distributed throughout the thickness of the scaffold.

In a more particular embodiment, the tissue comprises a scaffold which presents a first face comprising macropores of diameter comprised between 80 and 280 μm and a second opposite face comprising only nanopores of diameter below 10 nm, preferably below 5 nm.

In a preferred embodiment, said tissue is obtained after at least one week, preferably two, three or four weeks of culture.

Tissue obtained by the cell culture step as described above is thereafter tanned by any methods well-known in the art to create chemical bonds between the fibers of elastin and collagen to transform it into leather.

A variety of tanning processes may be used to tan leather, including chrome with chrome (III) sulfate tanning agent, tanning using aluminium salts, aldehydes, organic compounds, vegetable tanning using tannins, and tanning with phenolic type and acrylic type polymers of low molecular weight (hereafter “polymer tanning”).

In another aspect, the present disclosure relates to a tanned leather obtainable by the method as described above. As used herein, the term “leather” or “synthetic leather” refers to material obtained from the tanning or chemical treatment of animal skins or tissue comprising collagen, elastin and other components of the extracellular matrix. According to the present disclosure, said tissue (i.e. in vitro skin) is obtained by an in vitro culture step of fibroblasts and comprises a dense mesh of cells and extracellular matrix components.

The present disclosure relates to a tanned leather of in-vitro skin obtainable by the method of the present disclosure.

The present disclosure also relates to the use of macroporous scaffold as described above for producing leather, preferably by tanning tissue obtained from fibroblasts culture on said macroporous scaffold, more preferably wherein said scaffold comprises macropores of diameter comprised between 60 and 500 μm, preferably 80 μm and 400 μm, 100 and 350 μm, 100 and 300 μm, more preferably between 80 and 280 μm, 100 to 280 μm, again more preferably 120 to 250 μm. In a particular embodiment, said scaffold is a PDMS or polyester scaffold. In another particular embodiment, said scaffold is an asymmetric or symmetric scaffold as described above.

Embodiments of the present invention are described in the following specific examples which are exemplary only and not to be construed as limiting.

### EXAMPLES

In order to produce high quality leather, the inventors recreate skin in the laboratory. To do this the inventors follow three steps, the first is to create a scaffold that allows the cells to attach and develop. The second is to add cells to this scaffold, allowing them to proliferate and differentiate. The third is the implementation of a simplified tanning procedure of the material to transform the tissue into leather.

### 1. Scaffold

A great variety of scaffolds have been used in tissue engineering in the medical field. These scaffolds use different architectures, manufacturing methods, materials (biological and synthetic) and surface functionalization (Shafiee A, Atala A. Annu Rev Med. 2017 Jan 14;68:29-40). However, only few studies have been performed on the use of scaffold for tissue engineering in the field of leather synthesis.

The inventors decided to use a macroporous material for the production of the scaffold for the growth of cultured fibroblastic tissue in the laboratory.

Polymers with permeability to oxygen and/or small molecules to promote gas exchange, nutrients and the removal of waste associated with cell metabolism are required for the synthesis of fibroblastic tissue used in leather synthesis. For these reasons, the inventors have tested for the manufacture of porous scaffolds two families of polymers:
1. Polydimethylsiloxane (PDMS), a silicone elastomer most often used in microfluidic applications, as a polymer for creating a scaffold for leather due to its biocompatibility and long-term stability in contact with biological material (Aucoin L, et al. J Biomater Sci Polym Ed. 2002;13(4):447-62). Another important advantage of PDMS is the high solubility of oxygen in the PDMS matrix, which allows oxygen to be supplied to the cells inside the implants and to prevent hypoxia (Merkel, T.C., et al. (2000). J. Polym. Sci. B Polym. Phys., 38: 415-434). PDMS is hydrophobic, but its surface can be modified via methods such as plasma functionalization to make it hydrophilic. Once PDMS is hydrophilic, biological and/or chemical molecules can be attached, a necessary condition for cell adhesion (Li B, et al. J Biomed Mater Res A. 2006 Dec 15;79(4):989-98).
2. Biosourced, biodegradable and bioabsorbable polyesters and polyester derivatives are polymers that have interesting properties from a mechanical point of view, are compatible with cell culture in medical applications, are produced from agricultural waste and are fully biodegradable (Farah, Shady & Anderson, Daniel & Langer, Robert. (2016) Advanced Drug Delivery Reviews. 107. 10.1016/j.addr.2016.06.012; Kurokawa, N.; Kimura, S.; Hotta, A. J. Appl. Polym. Sci. 2018, 135, 45429; Meng, et al. (2016) Journal of Applied Polymer Science. 133. n/a-n/a. 10.1002/app.43530). However, they are impermeable to oxygen and do not allow the diffusion of molecules after reticulation.

To solve this, the inventors induced the formation of micro-pores of approximately 1-20 microns allowing increased diffusion of oxygen and molecules made using the “Non-solvent Induced Phase Separation Process” (NIPS) method as described by Al Tawil et al. 2018, European Polymer Journal. 105, 370-388, 2018.

The creation of macro-porous geometries (pores between 80-250 microns) on polymers based either on PDMS or polyesters was carried out using the “Solvent Casting and Particulate Leaching” (SCPL) method for the PDMS scaffolds and for the asymmetric polyester scaffolds, and NIPS coupled to SCPL for the symmetric polyester. In SCPL, the degree of porosity can be controlled by varying the percentage of particles relative to the solvent and the pore size is dictated by the diameter of the porogen in the solution (Sola A, et al. Mater Sci Eng C Mater Biol Appl. 2019), in NIPS the degree of porosity and the pore sizes are controlled by the physico-chemical properties of the macroporogen, the polymer/solvent mix and those of the non-solvent.

The inventors followed different methods for creating the macro-porous geometry depending on the polymer used as follows:
1. For PDMS, the inventor used SCPL method either by using size calibrated sodium chloride as described in Pedraza et al. 2012. J Biomater Sci Polym Ed. 2013;24(9):1041-56 or by adding a porogen with the correct particle size and highly soluble in water. The scaffold obtained is a symmetric scaffold with interconnected macropores of diameter between 150 and 250 μm distributed throughout the scaffold.
2. For polyester, the inventors used a combination of:
   a) One “Non-solvent Induced Phase Separation Process” (NIPS) process coupled with one “Solvent Casting and Particulate Leaching” (SCPL) process by spreading homogeneously the macro porogen particles on the support surface and pour the polymer/solvent mix over the macropore-containing support. The porous scaffolds obtained are symmetric with two porous faces containing macro and micropores.
   b) Using the SCPL method by adding a porogen with the correct particle size and highly soluble in water. The scaffold obtained is an asymmetric scaffold with a porous face containing interconnected macropores of diameter between 150 and 250 μm and smooth face without macro/micro pores.

Using the methods of creating porous geometries for PDMS and polyesters, the inventors obtained 2 different types of support symmetric or asymmetric scaffold (FIG. 1a-b).

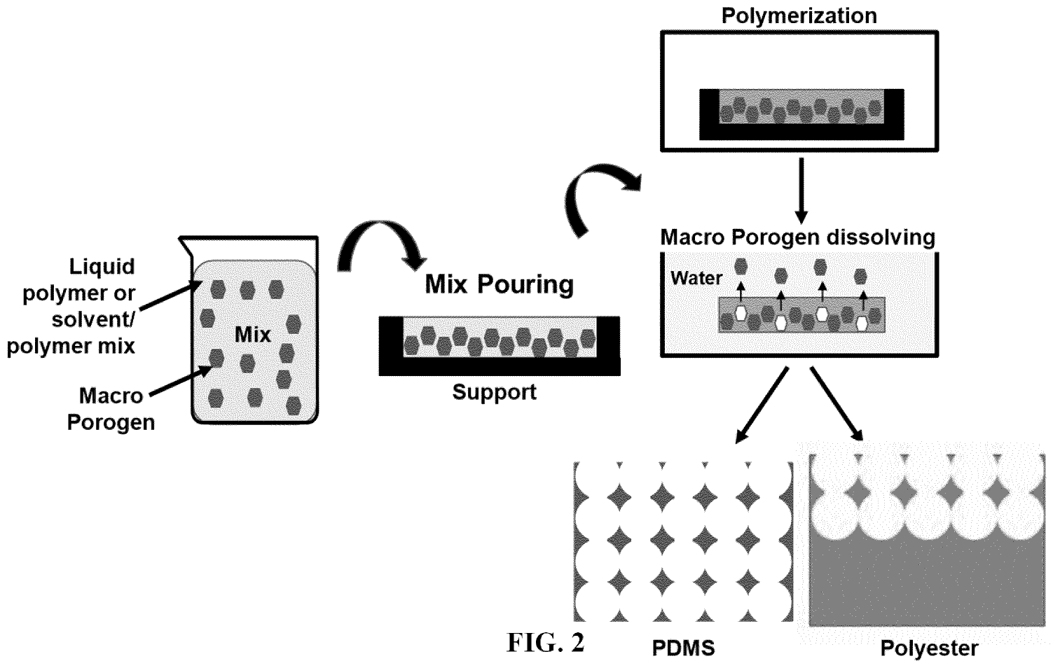

SCPL on PDMS and polyesters was performed as follows:
1. A symmetric support (FIG. 1a) in PDMS using the SCPL “Solvent Casting and Particulate Leaching” method (FIG. 2) was obtained by mixing the porogen and PDMS (Dow Sylgard 184 elastomer) and molding it on a support; the mixture was incubated at 50° C. for crosslinking of the polymer. The porogen was removed from the scaffolds by immersion in deionized water for 72 h, with water exchange every 24 h.
2. A symmetric support (FIG. 1a) in polyester using the Non-solvent Induced Phase Separation Process” (NIPS) method coupled to the SCPL “Solvent Casting and Particulate Leaching” method was produced by a series of steps detailed in FIG. 2. The “Non-solvent Induced Phase Separation Process” NIPS procedure was used to create micropores (up to 10 microns) non interconnected and parallel to the surface when the solvent/polymer mix was put in the non-solvent (water).
3. An asymmetric support (FIG. 1b) in polyester using the SCPL “Solvent Casting and Particulate Leaching” method was produced by a series of steps detailed in FIG. 2. The SCPL macropogen creates macropores (to around 120-160 μm) and induces macropore interconnectivity into the scaffold by dissolving into the non-solvent (water).

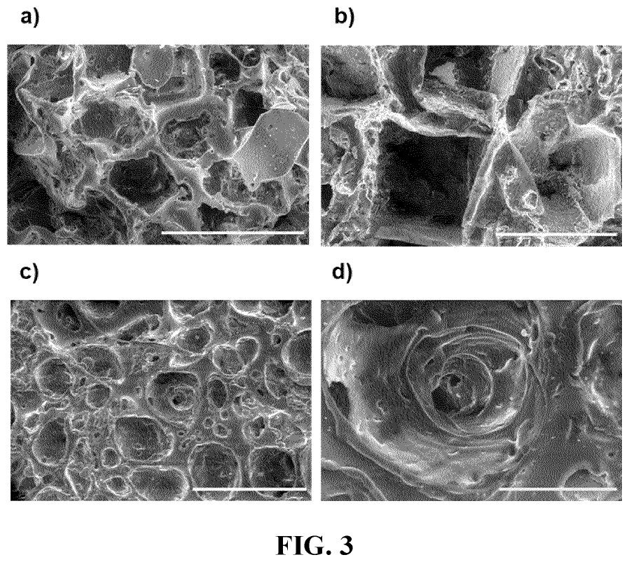

The physical and structural characteristics of the scaffolds were observed on a FEI Nova NanoSEM 450 model scanning electron microscope. Representative images of the surface condition of the scaffolds are shown on FIG. 3.

As shown in FIG. 3, PDMS scaffolds are porous on the surface with a range of square pore sizes varying from 100-200 microns for the mixtures containing PDMS with calibrated square macro porogens (FIG. 3a) and a more homogeneous distribution of round pore sizes with and approximate diameter of 160 microns for the mixtures with round shaped macro porogens (FIG. 3c-d). Higher magnification images in the panel 3d show that the pores are interconnected to the inside of the scaffold for the mixtures containing PDMS with both square and round shaped macro porogens (data not shown). The pixel-to-distance scale was obtained from the provided images (microns) and was used to determine the size of the pores using ImageJ (Rasband, W.S., ImageJ, U. S. National Institutes of Health, Bethesda, Maryland, USA, https://imagej.nih.gov/ij/, 1997-2018).

Polyester scaffolds showed a highly porous surface with pores sizes of an approximate diameter between 100-200 microns (FIG. 3b). The pixel-to-distance scale was obtained from the provided images (microns) and was used to determine the size of the pores using ImageJ (Rasband, W.S., ImageJ, U. S. National Institutes of Health, Bethesda, Maryland, USA, https://imagej.nih.gov/ij/, 1997-2018).

### 2. Functionalization

Two different physicochemical and chemical functionalization methods have been implemented for the PDMS or polyester scaffolds:

### 2.1. Physico-Chemical Method

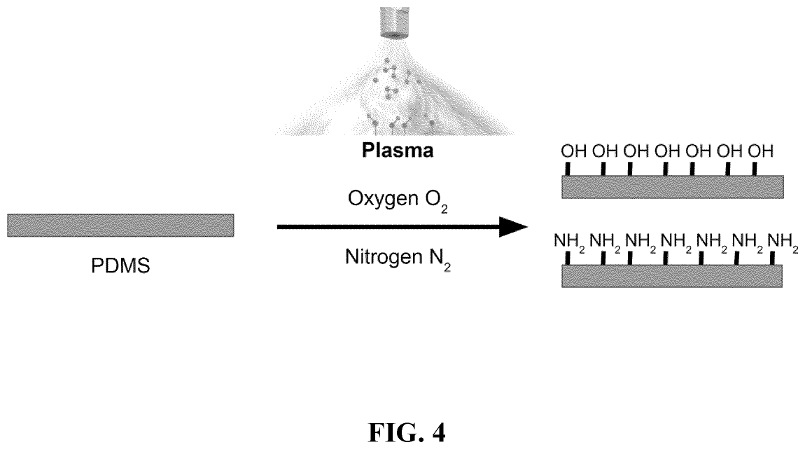

Physical functionalization using air/oxygen/nitrogen plasma (FIG. 4) has been adapted for PDMS scaffold from the methods currently used for the assembly of microfluidic chips (Tang, K.C. et al. (2006). Journal of Physics: Conference Series. 34. 155).

The procedure chosen for its efficiency was functionalization with plasma in a clean room equipped with plasma cleaning (Ref: PlasmaACE1 24 kHz, PLASMA Instrument).

Air/Oxygen plasma functionalization (FIG. 4) of PDMS adds electronegative silanol groups (Dhananjay Bodas, Chantal Khan-Malek. Chemical, Volume 123, Issue 1, 2007, Pages 368-373). Nitrogen functionalization (FIG. 4 ) of PDMS surfaces adds surface amine groups (Chengxin Yang and Yong J. Yuan. Applied Surface Science. Volume 364, 2016, Pages 815-821).

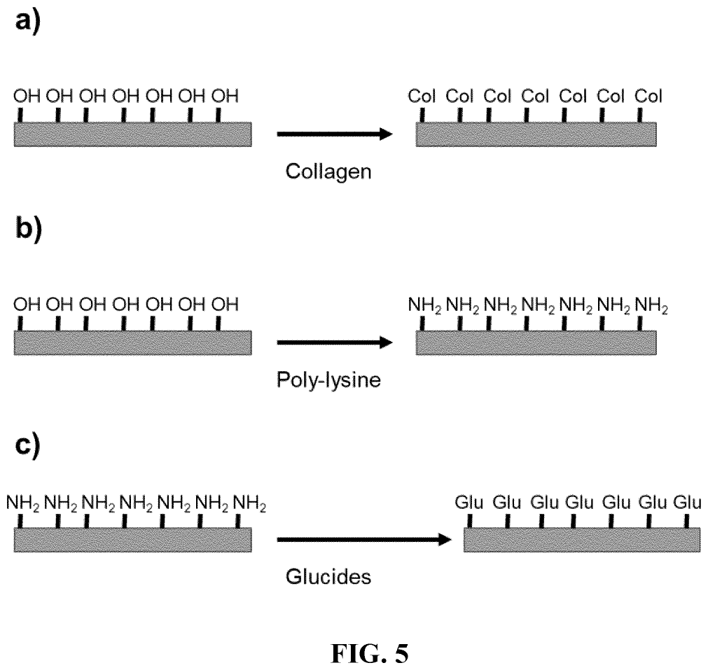

After plasma functionalization, the scaffolds were incubated immediately with different biological or chemical agents to functionalize its surface as follows:
1. Collagen (FIG. 5a): Incubation with 250 μg/mL of collagen I (50201, ibidi) for two hours. The excess collagen was removed by three successive washes with 15 ml of PBS, then incubated with 15 ml of culture media and inoculated with cells the same day.
2. Poly-lysine (FIG. 5b, left and center): Incubation with 50 mg/mL of poly-lysine (A3890401, Thermo) for two-three hours. The excess of poly-lysine was removed by three successive washes of deionized water, then incubated with culture media and inoculated with cells the same day.
3. Poly-lysine+mix of glucides/glucides derived molecules (FIG. 5c): Incubation with 50 mg/mL of poly-lysine (A3890401, Thermo) for two-three hours. The excess poly-lysine was removed by three successive washes with an excess of deionized water, then incubated with saturating concentration of a mix of glucides/glucides derived molecules. The excess of the functionalizing molecule was removed by three successive washes of deionized water, then incubated with culture media and inoculated with cells.
4. Glucides/glucides derived molecules (FIG. 5c): Incubation with saturating concentration of a mix of glucides/glucides derived molecules. The excess of the functionalizing molecule was removed by three successive washes of deionized water, then incubated culture media and inoculated with cells.

Functionalization with collagen adds a biologically active layer (FIG. 5a) that allows cell adhesion. Indeed, collagen specifically interacts with integrin receptors expressed at the surface of skin-derived cells (Cédric Zeltz, Donald Gullberg. J Cell Sci 15 February 2016; 129 (4): 653-664).

Air/Oxygen plasma functionalization (FIG. 4) of PDMS adds electronegative silanol groups on its surface that interact with positively charged (at physiological pH) amine groups of poly-lysine adding an overall positively charged layer at the surface of the PDMS scaffolds (FIG. 5b, left).

A layer of glucides/glucides derived molecules that facilitates cell adhesion, proliferation and collagen secretion, can be added to either: a) a poly-lysine coated PDMS scaffold (FIG. 5b), or b) a positively charged PDMS scaffold obtained by nitrogen plasma treatment (FIGS. 4 and 5c).

### 2.2 Chemical Method

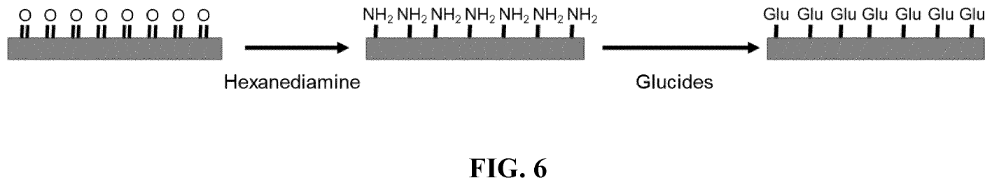

The functionalization of the entire surface of the porous polyester scaffolds was carried out according to a two-step process (FIG. 6). The aminolysis reaction of polyester scaffolds was carried out by immersing pieces of polyester scaffolds in solutions of hexane-1,6-diamine (HDA) dissolved in propanol/water mix for 15 minutes. The immobilization of glucides/glucides derived molecules on the surface of the polyester scaffolds was carried out at room temperature by immersing the aminolyzed polyester scaffolds in a solution containing the said glucides/glucides derived molecules.

### 3. Cell Culture on Scaffold

The skin is a flattened and extended anatomical structure that covers the entire external part of the body and continues with the mucous membranes at the level of the openings of the cavity's outwards. It is made up of three distinct layers: epidermis, dermis, and hypodermis. The epidermis is the outermost layer of the skin and is made up of a multi-layered, keratinized epithelium made up of multiple layers of cells. The cells that make up this layer are called keratinocytes and have a predominant function of protection against the environment.

The dermis is located below the epidermis and extends to the hypodermis and is made up of three main types of cells: a) Fibroblasts, the principal cell of the dermis, handle the synthesis of collagen, elastic and reticular fibers, and extracellular matrix material, and b) immune cells: histiocytes are tissue macrophages present within the connective tissue that assist the immune system. Mast cells are inflammatory cells located in the perivascular areas of the dermis (Brown TM, Krishnamurthy K. 2021 May 10. In: StatPearls [Internet]. Treasure Island (FL): StatPearls Publishing; 2021 Jan). In addition to these cells, the dermis is also made up of a complex and abundant material called the extracellular matrix (ECM) of which collagen and elastin, secreted by fibroblasts, are the major constituents (Schwarz R I. Biochem Biophys Rep. 2015 Sep;3:38-44).

With regard to the manufacture of classic cowhide leather, the tanning procedure completely removes the outer (epidermis) and inner (hypodermis) layers of the skin to keep only the dermis, which is the exclusive component of the leather in the final state (Sharphouse, J.H. Leather Technician's Handbook. Leather Producer's Association. P. 104. ISBN 0-9502285-1-6). Killing the animal and its subsequent storage dehydrates and breaks down most of the cellular, molecular and extracellular matrix components. The only components that resist storage and tanning are the collagen fibers, elastin fibers among other components of the extracellular matrix that are found in the leather in its final state. Collagen and elastin fibers are secreted by fibroblasts (Sharphouse, J.H. Leather Technician's Handbook. Leather Producer's Association. P. 104. ISBN 0-9502285-1-6). These collagen and elastin fibers provide the stiffness, mechanical strength and resistance to abrasion found in leather.

As the final cowhide leather contains only the dermis composed mostly of collagen and elastin fibers secreted by fibroblasts, the inventors decided to cultivate fibroblasts lines to obtain a dermal tissue particularly suitable for leather synthesis.

The fibroblasts are seeded onto the functionalized scaffold to allow them to proliferate under optimal conditions and induce the secretion of extracellular matrix (collagen, elastin among others).

### 3.1 Cell Growth Capacity

a. Biological Functionalization (Collagen)

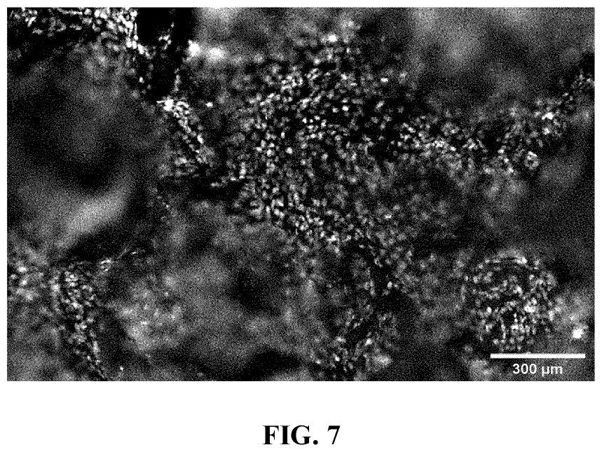

To explore cell growth and viability into the produced porous polymeric scaffolds functionalized with a biologically active molecule (biological functionalization), PDMS scaffolds (see FIG. 4a,c,d) functionalized with collagen I (FIG. 5a) and seeded with fibroblast cells for 7 days, fixed and stained with nucleus-marker DAPI staining protocol. Images of the seeded scaffolds were made by fluorescence microscopy, FIG. 7. As shown in FIG. 7, biological functionalization allowed us to attach cells at a very high density into the produced macroporous polymeric scaffolds.

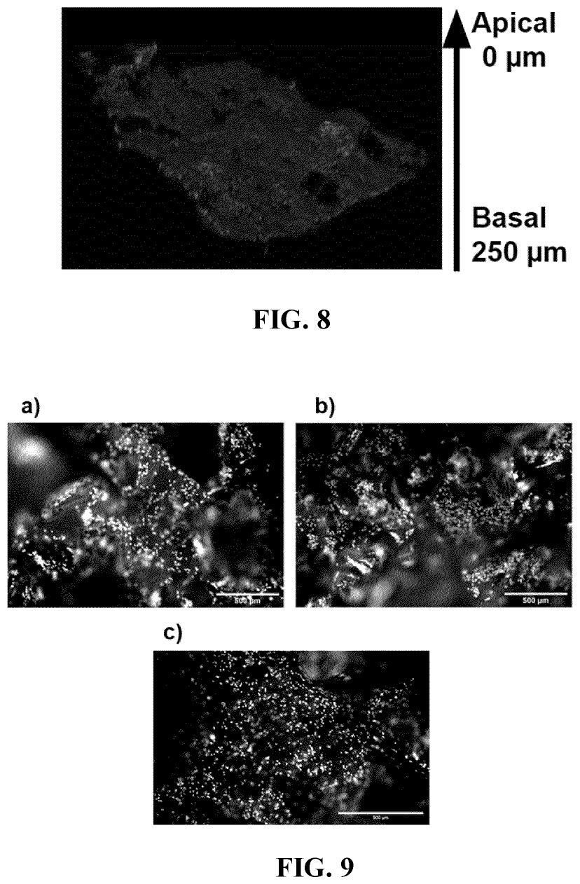

To explore cell growth and viability into the produced macroporous polymeric scaffolds for long periods of time. PDMS scaffolds functionalized with a biologically active molecule (collagen I) were seeded with fibroblast cells under standard culture conditions for 4 weeks, fixated and stained using the HCS LIVE/DEAD® Green Kit (Invitrogen, ref. H10290). The HCS LIVE/DEAD® Green Kit measures cytotoxicity using a non-fluorescent and cell impermeant compound that exhibits a strong fluorescent enhancement upon binding to DNA. Dead cells allow entry of the Image-iT® DEAD Green™ viability stain, while the assay also employs a cell permeant nuclear segmentation tool that stains both live and dead cells, reflecting the total cell number within the sample. The 4-week-stained samples were observed by confocal microscopy (OCCIGEN Imaging—Cytometry Platform, Genopole). FIG. 8 shows the high density of total cells while only few dead cells (<2%) were observed up to the basal 250 μm layer. These results showed that the PDMS scaffolds functionalized with collagen I (biological functionalization) allows efficient high cell attachment, cell culture and proliferation for long periods of time with minimal stress and damage for cells.

b. Chemical Functionalization (Poly-Lysine and Glucides/Glucides Derived Molecules, FIGS. 5b-c).

Cells were seeded on PDMS and polyester scaffolds coated with chemical surface functional layers (poly-lysine or glucides/glucides derived molecules) to assess the growth capacity of fibroblast cells using chemical molecules instead of collagen I or other biologically sourced proteins. Indeed, chemical functionalization with the molecules described above is several orders of magnitude less expensive than the use of biological origin proteins such as collagen or fibronectin.

The PDMS scaffolds functionalized with either poly-lysine or glucides/glucides derived molecules were seeded with fibroblast cells for 7 days, fixed and stained with nucleus-marker DAPI staining protocol. Images of the seeded scaffolds were made by fluorescence microscopy, FIG. 9a-b.

PDMS scaffolds functionalized with both poly-lysine (FIG. 9a) and glucides/glucides derived molecules (FIG. 9b) were both able to attach cells at a very high density as in the same order of magnitude observed in biologically functionalized scaffolds (Collagen I, FIG. 7).

Polyester scaffolds functionalized with either poly-lysine or glucides/glucides derived molecules were seeded with fibroblast cells for 7 days, fixed and stained with nucleus-marker DAPI staining protocol. Images of the seeded scaffolds were made by fluorescence microscopy, FIG. 9c. Polyester scaffolds functionalized with both poly-lysine (data not shown) or glucides/glucides derived molecules (FIG. 9c) were both able to attach cells at a very high density.

These results show efficient attachment, culture and proliferation of cells when using chemical agents for functionalization, opening the use of animal-free and economically viable reagents for the functionalization of the PDMS or polyester scaffolds.

### 3.2 Induction of Extracellular Matrix (ECM)

As explained above, the tanning procedure breaks down most of the cellular, molecular and extracellular matrix components. The only components that resist storage and tanning are mostly the collagen fibers and elastin fibers that are found in the leather in its final state and are secreted by fibroblasts (Sharphouse, J.H. Leather Technician's Handbook. Leather Producer's Association. P. 104. ISBN 0-9502285-1-6). These collagen and elastin fibers are responsible for the mechanical properties of leather. For this reason, inducing the secretion of Extracellular Matrix (collagen, elastin, among others) by the network of fibroblasts proliferating into the scaffolds is a key step in the development of leather.

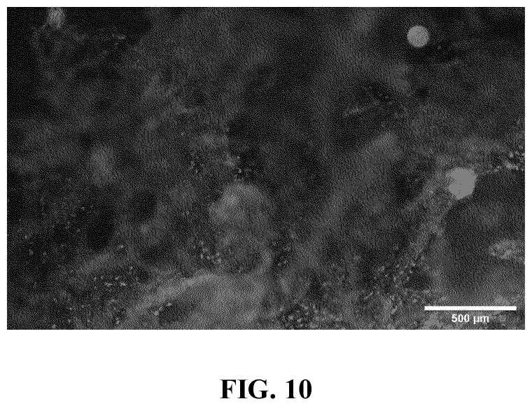

Collagen secretion experiments were performed to assess the ability of fibroblast cells to secrete extracellular matrix when seeded into our functionalized scaffolds. To assess this, fibroblast cells were seeded into a functionalized scaffold and cultivated for 4 weeks, fixed and co-stained with nucleus-marker DAPI and with an anti-collagen I antibody (Sigma, ref. C2456). Images of the seeded scaffolds were made by fluorescence microscopy, FIG. 10.

### 4. Tanning Step

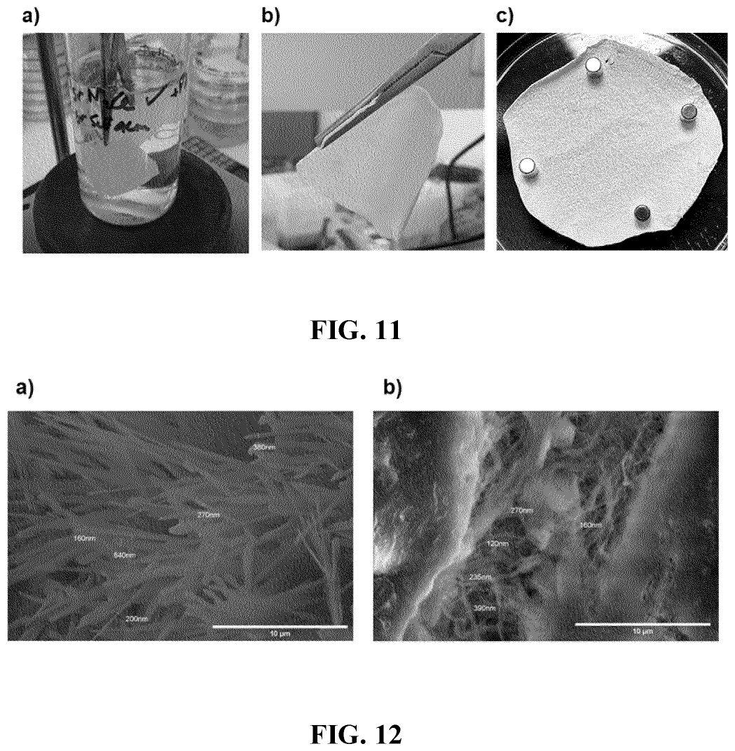

The tissue obtained after culture on the scaffold was submitted to a tanning process to obtain leather. The whole process can be divided into three different steps that are not forcefully applied to each type of leather (FIG. 11): a) simplified beamhouse operations process for soaking the tissue, eliminate the cell media and chemically prepare the tissue for tanning, b) tanning the tissue using any methods know by the art like chrome, aluminum, aldehyde, organic, synthetic and/or vegetable tanning to induce the formation of chemical links between the extracellular matrix chains in the tissue (FIG. 11a), and c) drying the tanned leather (FIG. 11b) to obtain the inventor's tanned leather (FIG. 11c).

The physical and structural characteristics of the extracellular matrix (ECM) collagen and elastin fibers of the obtained tanned leather are compared to those found in animal tanned leathers. The inventors observed their surface condition by scanning electron microscopy (SEM) on a FEI Nova NanoSEM 450 model scanning electron microscope and the scale was obtained from the provided images (microns) to determine the thickness of the ECM cables using ImageJ software. Images of the surface ECM fibers of inventor's tanned leather (FIG. 12a) and a German bull tanned leather (FIG. 12b) are shown on FIG. 12. The thickness of the extracellular matrix (ECM) collagen and elastin fibers of the rabbit leather obtained according to the present method is similar to that obtained in the high quality German bull tanned leather in an expected range between 150-750 microns.

1-16. (canceled)

17. A method for producing leather comprising the steps of:
a) culturing fibroblasts in vitro on a macroporous polymeric scaffold wherein said scaffold comprises macropores of diameter comprised between 60 and 500 μm. to obtain a tissue,
b) tanning said tissue thereby forming said leather.

18. The method of claim 17 wherein said scaffold comprises macropores of diameter comprised between 80 to 280 μm.

19. The method of claim 17 wherein said scaffold has at least one face with degree of porosity from 65% to 98%.

20. The method of claim 17 wherein said scaffold comprises at least one polymer.

21. The method of claim 17 wherein said scaffold comprises at least a polymer which is a polymeric organosilicon compound or biodegradable polyester.

22. The method of claim 17 wherein said scaffold comprises at least a polymer which is polydimethylsiloxane (PDMS).

23. The method according to claim 17 wherein said scaffold comprises at least a polymer which is polyester.

24. The method according to claim 17 wherein said scaffold comprises at least a polymer which is a biodegradable and bioresorbable polyester.

25. The method of claim 17 wherein said macropores are interconnected with micropores having a diameter of less than 20 μm.

26. The method according to claim 17 wherein said scaffold is a symmetric scaffold comprising macropores distributed over the thickness of the scaffold.

27. The method according to claim 26 wherein said scaffold has two macroporous faces, wherein said faces both comprise macropores of a diameter comprised between 60 and 500 μm.

28. The method of claim 25 wherein said scaffold comprises polydimethylsiloxane (PDMS) and wherein said scaffold is obtained by a solvent casting and particulate leaching (SCLP) method.

29. The method of claim 25 wherein said scaffold comprises polyester and wherein said scaffold is obtained by a solvent casting and particulate leaching (SCLP) method and a non-solvent induced phase separation process (NIPS).

30. The method according to claim 17 wherein said scaffold is an asymmetric scaffold comprising macropores of diameter comprised between 60 and 500 μm and micropores which have a diameter of less than 10 μm distributed throughout the thickness of the scaffold.

31. The method of claim 30 wherein said scaffold presents a first face comprising macropores of diameter comprised between 60 and 500 μm, and a second opposite face comprising only nanopores of diameter below 10 nm.

32. The method of claim 31 wherein said scaffold comprises a biodegradable polyester and wherein said scaffold is obtained by solvent casting and particulate leaching (SCLP).

33. The method according to claim 17 wherein a bioactive molecule is grafted at the surface of said scaffold.

34. The method according to claim 33 wherein said bioactive molecule is collagen, a glucide, or a molecule derived from a glucide.

35. A tissue obtainable after step a) of the method according to claim 17 comprising fibroblastic cells cultured on a macroporous scaffold as defined in claim 17.

36. A tanned leather obtainable by the method according to claim 17.
